(19) United States
(12) Patent Application Publication
(10) Pub. No.: US 2011/0060026 A1
(43) Pub. Date: Mar. 10, 2011

(54) Indoles Active on CRTH2 Receptor

(76) Inventors: George Hynd, Essex (GB); JOhn Gary Montana, Essex (GB); Harry Finch, Essex (GB)

(21) Appl. No.: 12/812,294

(22) PCT Filed: Jan. 16, 2009
(86) PCT No.: PCT/GB09/00124

§ 371 Date: Nov. 29, 2010

(30) Foreign Application Priority Data
Jan. 18, 2008 (GB) 0800961.5
Apr. 17, 2008 (GB) 0807047.6

(51) Int. Cl. A61K 31/405; C07D 209/24; A61P 11/06; A61P 11/00; A61P 17/06; A61P 17/00; A61P 1/00; A61P 1/04

(52) U.S. Cl. 514/419; 548/494

(57) ABSTRACT

Indole derivatives having therapeutic utility are of formula (I): X is —SO₂— or *—SO₂NR³— wherein the bond marked with an asterisk is attached to Ar¹; R¹ and R² are, independently, hydrogen, fluoro, chloro, CN or CF₃; R³ is hydrogen, C₁-C₈alkyl or C₃-C₇cycloalkyl; and Ar¹ and Ar² are, independently, phenyl or a 5- or 6-membered heteroaryl group, wherein the phenyl or heteroaryl group is optionally substituted by one or more substituents independently selected from fluoro, chloro, CN, C₃-C₇cycloalkyl, —O(C₁-C₄alkyl) or C₁-C₆alkyl, the latter two groups being optionally substituted by one or more fluoro atoms.

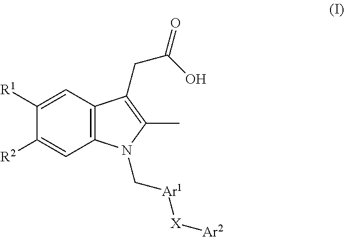

This invention relates to a class of indole compounds which are ligands of the CRTH2 receptor (Chemoattractant Receptor-homologous molecule expressed on T Helper cells type 2), and their use in the treatment of diseases responsive to modulation of CRTH2 receptor activity, principally diseases having a significant inflammatory component. The invention also relates to novel members of that class of ligands and pharmaceutical compositions containing them.

### BACKGROUND TO THE INVENTION

Mast cells are known to play an important role in allergic and immune responses through the release of a number of mediators, such as histamine, leukotrienes, cytokines, prostaglandin D₂, etc (Boyce; Allergy Asthma Proc., 2004, 25, 27-30). Prostaglandin D₂ (PGD₂) is the major metabolite produced by the action of cyclooxygenase on arachadonic acid by mast cells in response to allergen challenge (Lewis et al; J. Immunol., 1982, 129, 1627-1631). It has been shown that PGD₂ production is increased in patients with systemic mastocytosis (Roberts; N. Engl. J. Med., 1980, 303, 1400-1404), allergic rhinitis (Naclerio et al; Am. Rev. Respir. Dis., 1983, 128, 597-602; Brown et al. Arch. Otolarynol. Head Neck Surg., 1987, 113, 179-183; Lebel et al; J. Allergy Clin. Immunol., 1988, 82, 869-877), bronchial asthma (Murray et at N. Engl. J. Med., 1986, 315, 800-804; Liu et al; Am. Rev. Respir. Dis., 1990, 142, 126-132; Wenzel et at J. Allergy Clin. Immunol., 1991, 87, 540-548), and urticaria (Heavey et at J. Allergy Clin. Immunol., 1986, 78, 458-461). PGD₂ mediates it effects through two receptors, the PGD₂ (or DP) receptor (Boie et al; J. Biol. Chem., 1995, 270, 18910-18916) and the chemoattractant receptor-homologous molecule expressed on Th2 (or CRTH2) (Nagata et al; J. Immunol., 1999, 162, 1278-1289; Powell; Prostaglandins Luekot. Essent. Fatty Acids, 2003, 69, 179-185). Therefore, it has been postulated that agents that antagonise the effects of PGD₂ at its receptors may have beneficial effects in number of disease states.

The CRTH2 receptor has been shown to be expressed on cell types associated with allergic inflammation, such as basophils, eosinophils, and Th2-type immune helper cells (Hirai et al; J. Exp. Med., 2001, 193, 255-261). The CRTH2 receptor has been shown to mediate PGD₂-mediated cell migration in these cell types (Hirai et al; J. Exp. Med., 2001, 193, 255-261), and also to play a major role in neutrophil and eosinophil cell recruitment in a model of contact dermatitis (Takeshita et al; Int. Immunol., 2004, 16, 947-959). Ramatroban {(3R)-3-[(4-fluorophenyl)sulphonylamino]-1,2,3,4-tetrahydro-9H-carbazole-9-propanoic acid}, a dual CRTH2 and thromboxane A₂ receptor antagonist, has been shown to attenuate these responses (Sugimoto et al; J. Pharmacol. Exp. Ther., 2003, 305, 347-352; Takeshita et al; op. cit.). The potential of PGD₂ both to enhance allergic inflammation and induce an inflammatory response has been demonstrated in mice and rats. Transgenic mice over expressing PGD₂ synthase exhibit an enhanced pulmonary eosinophilia and increased levels of Th2 cytokines in response to allergen challenge (Fujitani et al, J. Immunol., 2002, 168, 443-449). In addition, exogenously administered CRTH2 agonists enhance the allergic response in sensitised mice (Spik et al; J. Immunol., 2005, 174, 3703-3708). In rats exogenously applied CRTH2 agonists cause a pulmonary eosinophilia but a DP agonist (BW 245C) or a TP agonist (1-BOP) showed no effect (Shirashi et al; J. Pharmacol. Exp Ther., 2005, 312, 954-960). These observations suggest that CRTH2 antagonists may have valuable properties for the treatment of diseases mediated by PGD₂.

In addition to Ramatroban a number of other CRTH2 antagonists have been described. Examples include: indoleacetic acids (WO2007/065684; WO2007/045867; WO2006/034419; WO2005/094816; WO2005/044260; WO2005/040114; WO2005/040112; GB2407318; WO2005/019171; WO2004/106302; WO2004/078719; WO2004/007451; WO2003/101981; WO2003/101961; WO2003/097598; WO2003/097042; WO2003/066047; WO2003/066046; WO2003/022813), quinolines (WO2007/036743), tetrahydroquinolines (WO2006/091674; US2005/256158; WO2005/100321; WO2005/007094; WO2004/035543; WO2004/032848; EP 1435356; EP1413306), phenoxyacetic acids (WO2007/062678; WO2007/062773; WO2006/125596; WO2006/125593; WO2006/056752; WO2005/115382; WO2005/105727; WO2005/018529; WO2004/089885; WO2004/089884) and phenylacetic acids (WO2004/058164).

### DETAILED DESCRIPTION OF THE INVENTION

One aspect of the invention provides indole derivatives of formula (I):

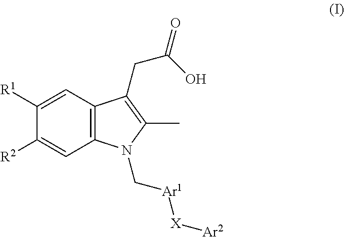

X is —SO₂— or *—SO₂NR³— wherein the bond marked with an asterisk is attached to Ar¹;

R¹ and R² are, independently, hydrogen, fluoro, chloro, CN or CF₃;

R³ is hydrogen, C₁-C₈alkyl or C₃-C₇cycloalkyl;

Ar¹ and Ar² are, independently, phenyl or 5- or 6-membered heteroaryl group, wherein the phenyl or heteroaryl groups are optionally substituted by one or more substituents independently selected from fluoro, chloro, CN, C₃-C₇cycloalkyl, —O(C₁-C₄alkyl) or C₁-C₆alkyl, the latter two groups being optionally substituted by one or more fluoro atoms.

Compounds (I) with which the invention is concerned are CRTH2 receptor antagonists, but they may also have beneficial effects at other prostanoid receptors, such as the PGD₂ receptor or the thromboxane A₂ receptor.

Compounds of formula (I) above may be prepared or recovered in the form of salts, and in some cases as N-oxides, hydrates, and solvates thereof. Any reference herein, including the claims herein, to “compounds of the invention”, “compounds with which the invention is concerned” or “compounds of formula (I)” and the like, includes reference to salts, particularly pharmaceutically acceptable salts, N-oxides, hydrates, and solvates of such compounds.

The invention also includes (i) use of a compound with which the invention is concerned in the manufacture of a medicament for use in the treatment of conditions responsive to modulation of CRTH2 receptor activity, and (ii) a method of treatment of conditions responsive to modulation of CRTH2 receptor activity, comprising administering to a patient suffering such disease an effective amount of a compound with which the invention is concerned.

Examples of conditions responsive to modulation of CRTH2 receptor activity include asthma, rhinitis, allergic airway syndrome, allergic rhinobronchitis, bronchitis, chronic obstructive pulmonary disease (COPD), nasal polyposis, sarcoidosis, farmer's lung, fibroid lung, cystic fibrosis, chronic cough, conjunctivitis, atopic dermatitis, Alzheimer's disease, amyotrophic lateral sclerosis, AIDS dementia complex, Huntington's disease, frontotemporal dementia, Lewy body dementia, vascular dementia, Guillain-Barre syndrome, chronic demyelinating polyradiculoneurophathy, multifocal motor neuropathy, plexopathy, multiple sclerosis, encephalomyelitis, panencephalitis, cerebellar degeneration and encephalomyelitis, CNS trauma, migraine, stroke, rheumatoid arthritis, ankylosing spondylitis, Behçet's Disease, bursitis, carpal tunnel syndrome, inflammatory bowel disease, Crohn's disease, ulcerative colitis, dermatomyositis, Ehlers-Danlos Syndrome (EDS), fibromyalgia, myofascial pain, osteoarthritis (OA), osteonecrosis, psoriatic arthritis, Reiter's syndrome (reactive arthritis), sarcoidosis, scleroderma, Sjogren's Syndrome, soft tissue disease, Still's Disease, tendinitis, polyarteritis Nodossa, Wegener's Granulomatosis, myositis (polymyositis dermatomyositis), gout, atherosclerosis, lupus erythematosus, systemic lupus erythematosus (SLE), type I diabetes, nephritic syndrome, glomerulonephritis, acute and chronic renal failure, eosinophilia fascitis, hyper IgE syndrome, sepsis, septic shock, ischemic reperfusion injury in the heart, allograft rejection after transplantations, and graft versus host disease.

However, the compounds with which the invention is concerned are primarily of value for the treatment of asthma, chronic obstructive pulmonary disease, rhinitis, allergic airway syndrome, or allergic rhinobronchitis. Psoriasis, atopic and non-atopic dermatitis Crohn's disease, ulcerative colitis, and irritable bowel disease are other specific conditions where the present compounds may have particular utility.

Another aspect of the invention is a pharmaceutical composition comprising a compound with which the invention is concerned in admixture with a pharmaceutically acceptable carrier or excipient.

### Terminology

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein the term “cycloalkyl” refers to a monocyclic saturated carbocyclic radical having from 3-8 carbon atoms and includes, for example, cyclopropyl, cyclobutyl, cyclopentyl, cyclohexyl, cycloheptyl and cyclooctyl.

As used herein the unqualified term “aryl” refers to a mono-, bi- or tri-cyclic carbocyclic aromatic radical, and includes radicals having two monocyclic carbocyclic aromatic rings which are directly linked by a covalent bond. Aryl radicals may have, for example, from 6 to 14 ring carbon atoms, preferably from 6 to 10 carbon atoms. Illustrative of aryl radicals are phenyl, biphenyl and napthyl.

As used herein the unqualified term “heteroaryl” refers to a mono-, bi- or tri-cyclic aromatic radical containing one or more heteroatoms selected from S, N and O, and includes radicals having two such monocyclic rings, or one such monocyclic ring and one monocyclic aryl ring, which are directly linked by a covalent bond. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyridazinyl, triazinyl, indolyl and indazolyl.

As used herein the term “salt” includes base addition, acid addition and quaternary salts. Compounds of the invention which are acidic can form salts, including pharmaceutically acceptable salts, with bases such as alkali metal hydroxides, for example sodium and potassium hydroxides; alkaline earth metal hydroxides, for example calcium, barium and magnesium hydroxides; with organic bases, for example N-methyl-D-glucamine, choline tris(hydroxymethyl)aminomethane, L-arginine, L-lysine, N-ethyl piperidine, dibenzylamine and the like. Specific salts with bases include the benzathine, calcium, diolamine, meglumine, olamine, potassium, procaine, sodium, tromethamine and zinc salts. Those compounds of the invention which are basic can form salts, including pharmaceutically acceptable salts with inorganic acids, for example with hydrohalic acids such as hydrochloric or hydrobromic acids, sulphuric acid, nitric acid or phosphoric acid and the like, and with organic acids, for example acetic, tartaric, succinic, fumaric, maleic, malic, salicylic, citric, methanesulphonic, p-toluenesulphonic, benzoic, benzenesulfonic, glutamic, lactic and mandelic acids and the like. Where a compound contains a quaternary ammonium group acceptable counter-ions may be, for example chlorides, bromides, sulfates, methanesulfonates, benzenesulfonates, toluenesulfonates (tosylates), napadisylates (naphthalene-1,5-disulfonates or naphthalene-1-(sulfonic acid)-5-sulfonates), edisylates (ethane-1,2-disulfonates or ethane-1-(sulfonic acid)-2-sulfonates), isethionates (2-hydroxyethylsulfonates), phosphates, acetates, citrates, lactates, tartrates, mesylates, maleates, malates, fumarates, succinates, xinafoates, p-acetamidobenzoates and the like; wherein the number of quaternary ammonium species balances the pharmaceutically acceptable salt such that the compound has no net charge.

Salts are discussed in the “Handbook of Pharmaceutical Salts. Properties, selection and use”, P. Heinrich Stahl & Camille G. Wermuth, Wiley-VCH, 2002.

The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Compounds with which the invention is concerned may exist in one or more stereoisomeric form, because of the presence of asymmetric atoms or rotational restrictions, and in such cases can exist as a number of stereoisomers with R or S stereochemistry at each chiral centre or as atropisomers with R or S stereochemistry at each chiral axis. The invention includes all such enantiomers and diastereoisomers and mixtures thereof.

Use of prodrugs, such as esters, of compounds with which the invention is concerned is also part of the invention. “Prodrug” means a compound which is convertible in vivo by metabolic means (for example, by hydrolysis, reduction or oxidation) to a compound of formula (I). For example an ester prodrug of a compound of formula (I) may be convertible by hydrolysis in vivo to the parent molecule. Suitable esters of compounds of formula (I) are for example acetates, citrates, lactates, tartrates, malonates, oxalates, salicylates, propionates, succinates, fumarates, maleates, methylene-bis-β-hydroxynaphthoates, gentisates, isethionates, di-p-toluoyltartrates, methanesulphonates, ethanesulphonates, benzenesulphonates, p-toluenesulphonates, cyclohexylsulphamates and quinates. Examples of ester prodrugs are those described by F. J. Leinweber, Drug Metab. Res., 1987, 18, 379. As used in herein, references to the compounds of formula (I) are meant to also include the prodrug forms.

Structural Aspects of Compounds with which the Invention is Concerned

R¹ and R² are, independently, hydrogen, fluoro, chloro, CN or CF₃. In one subset of compounds of the invention R¹ is fluoro and R² is hydrogen. In another subset of compounds of the invention R¹ is hydrogen and R² is fluoro. All combinations of the permitted substituents R¹ and R² are allowed.

Ar¹ and Ar² are, independently, phenyl or 5- or 6-membered heteroaryl. Examples of such rings include phenyl, pyrrole, imidazole, furan, thiophene, oxazole, thiazole, pyrazole, isoxazole, isothiazole, pyridine, pyridazine, pyrimidine and pyrazine. Currently preferred is the case where both Ar¹ and Ar¹ are phenyl rings.

Ar¹ and Ar² may be optionally be substituted by one or more substituents independently selected from fluoro, chloro, CN, C₃-C₇cycloalkyl such as cyclopropyl, —O(C₁-C₄alkyl) such as methoxy, C₁-C₆alkyl such as methyl or the latter two groups being optionally substituted by one or more fluoro atoms, as in the case of trifluormethoxy or trifluoromethyl.

Currently it is preferred that the radical Ar²X— is in the para-, or more preferably the ortho-position of the ring Ar¹ relative to the point of attachment of Ar¹ to the rest of the molecule.

Specific compounds of the invention include those of the Examples herein.

### Compositions

As mentioned above, the compounds with which the invention is concerned are CRTH2 receptor antagonists, and are useful in the treatment of diseases which benefit from such modulation. Examples of such diseases are referred to above, and include asthma, rhinitis, allergic airway syndrome, bronchitis and chronic obstructive pulmonary disease.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial, as is required in the pharmaceutical art. In general, the daily dose range will lie within the range of from about 0.001 mg to about 100 mg per kg body weight of a mammal, often 0.01 mg to about 50 mg per kg, for example 0.1 to 10 mg per kg, in single or divided doses. On the other hand, it may be necessary to use dosages outside these limits in some cases.

The compounds with which the invention is concerned may be prepared for administration by any route consistent with their pharmacokinetic properties. Orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulfate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

The drug may also be formulated for inhalation, for example as a nasal spray, or dry powder or aerosol inhalers. For delivery by inhalation, the active compound is preferably in the form of microparticles. They may be prepared by a variety of techniques, including spray-drying, freeze-drying and micronisation. Aerosol generation can be carried out using, for example, pressure-driven jet atomizers or ultrasonic atomizers, preferably using propellant-driven metered aerosols or propellant-free administration of micronized active compounds from, for example, inhalation capsules or other “dry powder” delivery systems.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

Other compounds may be combined with compounds with which the invention is concerned for the prevention and treatment of prostaglandin-mediated diseases. Thus the present invention is also concerned with pharmaceutical compositions for preventing and treating PGD₂-mediated diseases comprising a therapeutically effective amount of a compound of the invention and one or more other therapeutic agents. Suitable therapeutic agents for a combination therapy with compounds of the invention include, but are not limited to: (1) corticosteroids, such as fluticasone, ciclesonide or budesonide; (2) β2-adrenoreceptor agonists, such as salmeterol, indacaterol or formoterol; (3) leukotriene modulators, for example leukotriene antagonists such as montelukast, zafirulast or pranlukast or leukotriene biosynthesis inhibitors such as Zileuton or BAY-1005; (4) anticholinergic agents, for example muscarinic-3 (M3) receptor antagonists such as tiotropium bromide; (5) phosphodiesterase-IV (PDE-IV) inhibitors, such as roflumilast or cilomilast; (6) antihistamines, for example selective histamine-1 (H1) receptor antagonists, such as fexofenadine, citirizine, loratidine or astemizole; (7) antitussive agents, such as codeine or dextramorphan; (8) non-selective COX-1/COX-2 inhibitors, such as ibuprofen or ketoprofen; (9) COX-2 inhibitors, such as celecoxib and rofecoxib; (10) VLA-4 antagonists, such as those described in WO97/03094 and WO97/02289; (11) TACE inhibitors and TNF-α inhibitors, for example anti-TNF monoclonal antibodies, such as Remicade and CDP-870 and TNF receptor immunoglobulin molecules, such as Enbrel; (12) inhibitors of matrix metalloprotease, for example MMP12; (13) human neutrophil elastase inhibitors, such as those described in WO2005/026124, WO2003/053930 and WO06/082412; (14) A2a agonists such as those described in EP1052264 and EP1241176 (15) A2b antagonists such as those described in WO2002/42298; (16) modulators of chemokine receptor function, for example antagonists of CCR3 and CCR8; (17) compounds which modulate the action of other prostanoid receptors, for example a thromboxane A₂ antagonist; and (18) agents that modulate Th2 function, such as PPAR agonists.

The weight ratio of the compound of the invention to the second active ingredient may be varied and will depend upon the effective dose of each ingredient. Generally, an effective dose of each will be used.

### Synthesis

There are multiple synthetic strategies for the synthesis of the compounds with which the present invention is concerned, but all rely on known chemistry, known to the synthetic organic chemist. Thus, compounds of the invention can be synthesised according to procedures described in the standard literature and are well-known to the one skilled in the art. Typical literature sources are “Advanced organic chemistry”, 4^{th} Edition (Wiley), J. March, “Comprehensive Organic Transformation”, 2^{nd} Edition (Wiley), R. C. Larock, “Handbook of Heterocyclic Chemistry”, 2^{nd} Edition (Pergamon), A. R. Katritzky, review articles such as found in “Synthesis”, “Acc. Chem. Res.”, “Chem. Rev.”, or primary literature sources identified by standard literature searches online or from secondary sources such as “Chemical Abstracts” or “Beilstein”. The extensive literature relating to the synthesis of indole compounds is especially relevant, of course.

It may be necessary to protect reactive functional groups (for example, hydroxy, amino, thio or carboxy) in intermediates used in the preparation of compounds of formula (I) to avoid their unwanted participation in a reaction leading to the formation of compounds of formula (I). Conventional protecting groups, for example those described by T. W. Greene and P. G. M. Wuts in “Protective groups in organic chemistry” John Wiley and Sons, 1999, may be used.

The compounds of the invention of formula (I) may be isolated in the form of their pharmaceutically acceptable salts, such as those described previously herein above. The free acid form corresponding to isolated salts can be generated by acidification with a suitable acid such as acetic acid and hydrochloric acid and extraction of the liberated free acid into an organic solvent followed by evaporation. The free acid form isolated in this manner can be further converted into another pharmaceutically acceptable salt by dissolution in an organic solvent followed by addition of the appropriate base and subsequent evaporation, precipitation, or crystallisation.

Compounds of formula (Ia), wherein X, R¹, R², Ar¹ and Ar² are as defined for formula (I) above, may be prepared by the reaction between an indole of formula (II), wherein E represents hydrogen or alkyl group, and a suitable alkylating agent of formula (III), wherein LG represents a suitable leaving group (for example, chloro, bromo or methanesulfonyloxy) (Scheme 1). Typically, the alkylation reaction is carried out in the presence of base (for example, sodium hydride or potassium carbonate) in an inert solvent (for example, dimethyl sulfoxide or N,N-dimethylformamide). It is to be understood that if the reaction is carried out on a protected form of (II) an appropriate deprotection step will be required to obtain the desired compound of the invention (Ia).

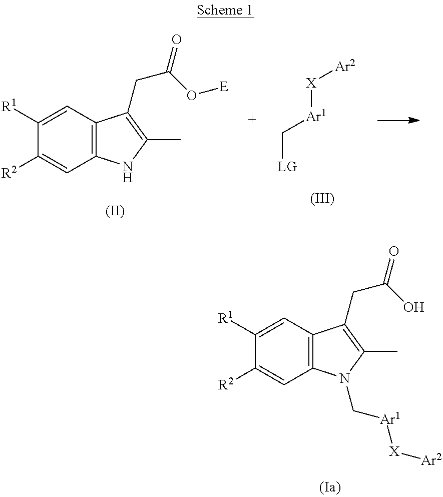

Compounds of formula (II) are commercially available or can be prepared by known methods. Compounds of formula (III) may be prepared by the reduction of compounds of formula (V) to an intermediate alcohol of formula (IV), followed by reaction with an appropriate halogenating (for example, phosphorus tribromide) or triflating (for example, triflic anhydride) agent (Scheme 2).

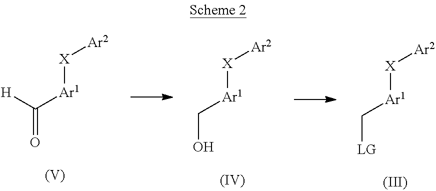

Compounds of formula (Va), wherein X represents SO₂ group, may be prepared by the oxidation of compounds of formula (VI), with a suitable oxidising agent such as potassium peroxymonosulfate, meta-chloroperoxybenzoic acid or other well known oxidising agents (Scheme 3).

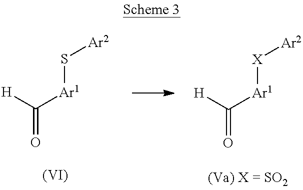

Compounds of formula (VI) may be prepared from intermediate compounds of formula (VII), wherein T represents a chloro, bromo, or iodo atom, or a trifluoromethanesulfonyloxy group, by reaction with a thiol of formula (VIII) in the presence of a suitable base such as potassium carbonate (Scheme 4). Alternatively, the reaction may be carried out in the presence of a suitable catalyst, such as tetrakis(triphenylphosphine)palladium(0) in a protic solvent such as ethanol. Compounds of formula (VII) and (VIII) are commercially available or can be prepared by known methods.

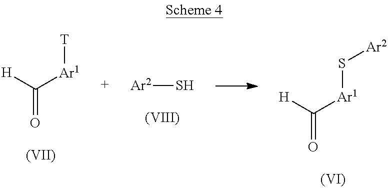

Compounds of formula (Vb), wherein X represents SO₂NR³ group, may be prepared by the reaction between a compound of formula (IX) and an amine of formula (X) (Scheme 5). The reaction may be carried out in the presence of a suitable base (for example, triethylamine or diisopropylethylamine) and solvent (for example, dichloromethane or dichloroethane), at temperatures ranging from 0° C. to the reflux temperature of the solvent, preferably at about room temperature. Compounds of formula (IX) and (X) are commercially available or can be prepared by known methods.

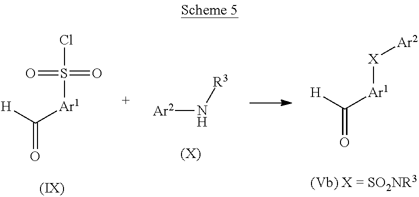

Alternatively, intermediate compounds of formula (III), wherein LG represents a chloro or bromo group, may be prepared from compounds of formula (XI) by treatment with N-chlorosuccinimide or N-bromosuccinimide in the presence of a suitable radical initiator (for example, 2,2′-azobisisobutyronitrile or benzoyl peroxide) (Scheme 6).

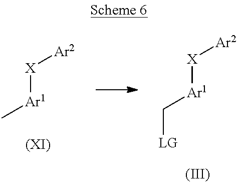

Compounds of formula (XIa), wherein X represents SO₂ group, may be prepared from compounds of formula (VIII) and (XII) (Scheme 7), using methods described above for the preparation of compounds of formula (Va) from compounds of formula (VII) and (VIII) (Schemes 3 and 4). Compounds of formula (XII) are commercially available or can be prepared by known methods.

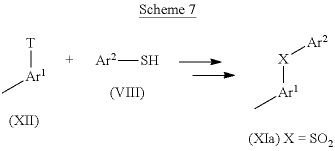

Compounds of formula (XIb), wherein X represents SO₂NR³ group, may be prepared from compounds of formula (X) and (XIII) (Scheme 8), using methods described above for the preparation of compounds of formula (Vb) from compounds of formula (X) and (XI) (Scheme 5). Compounds of formula (XIII) are commercially available or can be prepared by known methods.

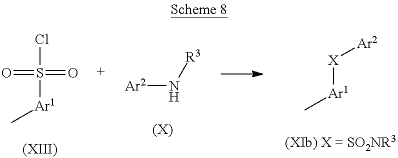

### EXAMPLES

¹H NMR spectra were recorded at ambient temperature using a Varian Unity Inova (400 MHz) spectrometer with a triple resonance 5 mm probe spectrometer. Chemical shifts are expressed in ppm relative to tetramethylsilane. The following abbreviations have been used: br s=broad singlet, s=singlet, d=doublet, dd=double doublet, t=triplet, q=quartet, m=multiplet.

Mass Spectrometry (LCMS) experiments to determine retention times and associated mass ions were performed using the following methods:

Method A: experiments were performed on a Micromass Platform LCT spectrometer with positive ion electrospray and single wavelength UV 254 nm detection using a Higgins Clipeus C18 5 μm 100×3.0 mm column and a 2 mL/minute flow rate. The initial solvent system was 95% water containing 0.1% formic acid (solvent A) and 5% acetonitrile containing 0.1% formic acid (solvent B) for the first minute followed by a gradient up to 5% solvent A and 95% solvent B over the next 14 minutes. The final solvent system was held constant for a further 2 minutes.

Method B: experiments were performed on a Micromass Platform LC spectrometer with positive and negative ion electrospray and ELS/Diode array detection using a Phenomenex Luna C18(2) 30×4.6 mm column and a 2 mL/minute flow rate. The solvent system was 95% solvent A and 5% solvent B for the first 0.50 minutes followed by a gradient up to 5% solvent A and 95% solvent B over the next 4 minutes. The final solvent system was held constant for a further 0.50 minutes

### Example 1

### {5-fluoro-1-[2-(4-fluorobenzenesulfonyl)benzyl]-2-methyl-1H-indol-3-yl}acetic acid

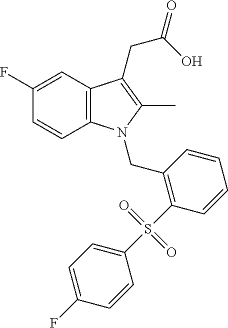

### Preparation 1a: 2-(4-fluorobenzenesulfonyl)benzaldehyde

A solution of 2-(4-fluorophenylsulfanyl)benzaldehyde (1.0 g) in dichloromethane (43 mL) was treated portionwise with 3-chloroperoxybenzoic acid (2.9 g), and the resulting mixture was stirred at room temperature for 2 hours. The mixture was diluted with saturated aqueous sodium thiosulphate solution and extracted with diethyl ether (3×50 mL). The combined extracts were washed with saturated aqueous sodium hydrogen carbonate solution (3×25 mL) and saturated aqueous sodium chloride solution (20 ml), and then dried over magnesium sulfate. The solvent was removed under reduced pressure to afford the title compound as a white solid (0.89 g).

¹H NMR (CDCl₃): δ 7.22 (m, 2H), 7.72-7.80 (m, 2H), 7.93 (m, 2H), 8.03 (m, 1H), 8.17 (m, 1H), 10.84 (s, 1H).

### Preparation 1b: [2-(4-fluorobenzenesulfonyl)phenyl]methanol

A solution of 2-(4-fluorobenzenesulfonyl)benzaldehyde (0.89 g) in methanol (34 mL) at 0° C. was treated with sodium borohydride (0.038 g), and the resulting mixture was stirred at room temperature for 1 hour. The mixture was diluted with 1.0 M aqueous hydrochloric acid (3.0 mL) and concentrated under reduced pressure. The residue was diluted with diethyl ether, washed with water (2×20 mL) and saturated aqueous sodium chloride solution (10 mL), and then dried over magnesium sulfate. The solvent was removed under reduced pressure to afford title compound as a colourless oil (0.89 g).

¹H NMR (CDCl₃): δ 2.99 (br s, 1H), 4.76 (d, J=5.0, 2H), 7.20 (m, 2H), 7.52 (ddd, J=1.5, 7.8, 7.8 Hz, 2H), 7.58 (dd, J=1.3, 7.5 Hz, 1H), 7.64 (ddd, J=1.4, 7.5, 7.5 Hz, 1H), 7.92 (m, 2H), 8.10 (dd, J=1.3, 7.9 Hz, 1H).

### Preparation 1c: Preparation of 2-(4-fluorobenzenesulfonyl)benzyl bromide

A solution of [2-(4-fluorobenzenesulfonyl)phenyl]methanol (0.86 g) in dichloromethane (16 mL) at 0° C. was treated with phosphorus tribromide (0.30 mL), and the resulting mixture was stirred at room temperature for 10 minutes. The mixture was poured into a mixture of saturated aqueous sodium thiosulphate solution (30 mL) and saturated aqueous sodium chloride solution (20 mL). The aqueous phase was extracted with dichloromethane (2×20 mL), and the combined organic phases were dried over magnesium sulfate and concentrated under reduced pressure. The residue was purified by column chromatography on silica gel, eluting with a mixture of cyclohexane and ethyl acetate (1:0 to 3:2 by volume) to afford the title compound as a colourless oil (0.27 g).

¹H NMR (CDCl₃): δ 4.89 (s, 2H), 7.20 (m, 2H), 7.51 (ddd, J=1.7, 7.1, 8.3 Hz, 1H), 7.57 (dd, J=1.7, 7.8 Hz, 1H), 7.61 (ddd, J=1.7, 7.0, 7.8 Hz, 1H), 7.94 (m, 2H), 8.17 (dd, J=1.4, 8.0 Hz, 1H).

### Preparation 1d: {5-fluoro-1-[2-(4-fluorobenzenesulfonyl)benzyl]-2-methyl-1H-indolyl}acetic acid ethyl ester

A solution of (5-fluoro-2-methyl-1H-indol-3-yl)acetic acid ethyl ester (0.064 g) in dimethyl sulfoxide (1.4 mL) was treated with sodium hydride (60% slurry in mineral oil, 0.016 g), and the resulting mixture was stirred at room temperature for 30 minutes. The mixture was treated with 2-(4-fluorobenzenesulfonyl)benzyl bromide (0.13 g) and potassium iodide (0.068 g), and the resulting mixture was stirred at room temperature for 12 hours, and then diluted with saturated aqueous ammonium chloride solution (2.0 mL) and water (25 mL). The mixture was extracted with ethyl acetate (3×10 mL), and the combined extracts were washed with saturated aqueous sodium chloride solution (2.0 mL) and dried over magnesium sulphate. The solvent was removed under reduced pressure, and the residue purified by column chromatography on silica gel, eluting with a mixture of cyclohexane and ethyl:acetate (1:0 to 3:2 by volume) to afford the title compound as a colourless oil (0.079 g).

MS: ESI (+ve) (Method B): 484 (M+H)⁺, Retention time 4.3 min.

¹H NMR (CDCl₃): δ 1.24 (t, J=7.1 Hz, 3H), 2.05 (s, 3H), 3.65 (s, 2H), 4.13 (q, J=7.1 Hz, 2H), 5.48 (s, 2H), 6.27 (d, J=7.8 Hz, 1H), 6.52 (dd, J=4.1, 8.8 Hz, 1H), 6.73 (ddd, J=2.4, 9.0, 9.0 Hz, 1H), 7.21 (dd, J=2.3, 9.4 Hz, 1H), 7.28 (m, 2H), 7.37 (ddd, J=1.4, 7.7, 7.7 Hz, 1H), 7.45 (dd, J=7.7, 7.7, 1.2 Hz, 1H), 8.00 (m, 2H), 8.21 (dd, J=7.8, 1.4 Hz, 1H).

### Preparation 1e: {5-fluoro-1-[2-(4-fluorobenzenesulfonyl)benzyl]-2-methyl-1H-indol-3-yl}acetic acid

A solution of {5-fluoro-1-[2-(4-fluorobenzenesulfonyl)benzyl]-2-methyl-1H-indolyl}acetic acid ethyl ester (0.041 g) in tetrahydrofuran (1.4 mL) was treated with 2.0 M aqueous lithium hydroxide solution (1.4 mL), and the resulting mixture was stirred at room temperature for 17 hours. The mixture was diluted with water (10 mL), pH adjusted to 4 by the addition of 1.0 M aqueous hydrochloric acid and extracted with ethyl acetate (3×10 mL). The combined extracts were washed with saturated aqueous sodium chloride solution (2.0 mL) and dried over magnesium sulfate. The solvent was removed under reduced pressure, and the residue purified by preparative reverse-phase HPLC to afford the title compound as a white solid (0.023 g).

MS: ESI (+ve) (Method A): 456 (M+H)⁺, Retention time 11.2 min.

¹H NMR (DMSO-d₆): δ 1.91 (s, 1H), 3.56 (s, 2H), 5.48 (s, 2H), 6.13 (d, J=7.5 Hz, 1H), 6.66-6.75 (m, 2H), 7.19 (dd, J=2.2, 10.0 Hz, 1H), 7.46-7.57 (m, 4H), 8.11 (m, 2H), 8.17 (dd, J=1.4, 7.7 Hz, 1H), 12.14 (br s, 1H).

### Example 2

### {6-fluoro-1-[2-(4-fluorobenzenesulfonyl)benzyl]-2-methyl-1H-indol-3-yl}acetic acid

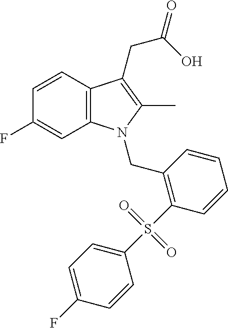

### Preparation 2a: (6-fluoro-2-methyl-1H-indol-3-yl)acetic acid ethyl ester

A mixture of (3-fluorophenyl)hydrazine hydrochloride salt (3.5 g), 4-oxo-pentanoic acid (2.3 g) and ethanol (24 mL) was treated with sulfuric acid (2.0 mL), and the resulting reaction mixture was stirred at 85° C. for 3 days. The mixture was cooled to room temperature, poured onto a mixture of ice and water and extracted with dichloromethane (3×50 mL). The combined extracts were washed with saturated aqueous sodium chloride solution (2.0 mL) and dried over magnesium sulfate. The solvent was removed under reduced pressure, and the residue purified by column chromatography on silica gel, eluting with a mixture of cyclohexane, ethyl acetate and formic acid (1:0:0.001 to 0:1:0.001 by volume) to afford the title compound as a yellow oil (0.40 g).

MS: ESI (+ve) (Method B): 236 (M+H)⁺, Retention time 3.5 min.

### Preparation 2b: {6-fluoro-1-[2-(4-fluorobenzenesulfonyl)benzyl]-2-methyl-1H-indol-3-yl}acetic acid ethyl ester

The title compound was prepared by the method of Preparation 1d using (6-fluoro-2-methyl-1H-indol-3-yl)acetic acid ethyl ester and 2-(4-fluorobenzenesulfonyl)benzyl bromide.

MS: ESI (+ve) (Method B): 484 (M+H)⁺, Retention time 4.2 min.

¹H NMR (CDCl₃): δ 1.23 (t, J=7.1 Hz, 3H), 2.07 (s, 3H), 3.67 (s, 2H), 4.14 (q, J=7.1 Hz, 2H), 5.43 (s, 2H), 6.17 (dd, J=2.1, 9.7 Hz, 1H), 6.33 (dd, J=1.0, 7.7 Hz, 1H), 6.83 (dt, J=2.2, 8.5 Hz, 1H), 7.38 (m, 5H), 8.00 (m, 2H), 8.24 (dd, J=1.3, 7.7 Hz, 1H) 12.6 (br s, 1H).

### Preparation 2c: {6-fluoro-1-[2-(4-fluorobenzenesulfonyl)benzyl]-2-methyl-1H-indol-3-yl}acetic acid

A solution of {6-fluoro-1-[2-(4-fluorobenzenesulfonyl)benzyl]-2-methyl-1H-indolyl}acetic acid ethyl ester (0.095 g) in tetrahydrofuran (5.0 mL) was treated with 1.0 M aqueous lithium hydroxide solution (0.5 mL), and the resulting mixture was stirred at room temperature for 22 hours. The mixture was diluted with water (10 mL), pH adjusted to 5 by the addition of 1.0 M aqueous hydrochloric acid and extracted with ethyl acetate (3×10 mL). The combined extracts were washed with saturated aqueous sodium chloride solution (2.0 mL) and dried over magnesium sulfate. The solvent was removed under reduced pressure, and the residue purified by column chromatography on silica gel, eluting with a mixture of dichloromethane, ethyl acetate and formic acid (1:0:0.001 to 0:1:0.001 by volume) to afford the title compound as a white solid (0.067 g).

MS: ESI (+ve) (Method A): 456 (M+H)⁺, Retention time 11.2 min.

¹H NMR (DMSO-d₆): δ 1.94 (s, 3H), 3.61 (s, 2H), 5.49 (s, 2H), 6.22 (d, J=7.4 Hz, 1H), 6.46 (dd, J=2.2, 10.2 Hz, 1H), 6.83 (m, 1H), 7.44 (dd, J=5.5, 8.7 Hz, 1H), 7.54 (m, 4H), 8.16 (m, 2H), 8.24 (dd, J=1.4, 7.7 Hz, 1H).

### Biological Methods

Compounds of the invention were tested using the following biological test method to determine their ability to displace PGD₂ from the CRTH2 receptor.

### CRTH2 Radioligand Binding Assay

The receptor binding assay is performed in a final volume of 200 μL binding buffer [10 mM BES (pH 7.4), 1 mM EDTA, 10 mM manganese chloride, 0.01% BSA] and 1 nM [³H]-PGD₂ (Amersham Biosciences UK Ltd). Ligands are added in assay buffer containing a constant amount of DMSO (1% by volume). Total binding is determined using 1% by volume of DMSO in assay buffer and non-specific binding is determined using 10 μM of unlabeled PGD₂ (Sigma). Human embryonic kidney (HEK) cell membranes (3.5 μg) expressing the CRTH2 receptor are incubated with 1.5 mg wheatgerm agglutinin SPA beads and 1 nM [³H]-PGD₂ (Amersham Biosciences UK Ltd) and the mixture incubated for 3 hours at room temperature. Bound [³H]-PGD₂ is detected using a Microbeta TRILUX liquid scintillation counter (Perkin Elmer). Compound IC₅₀ value is determined using a 6-point dose response curve in duplicate with a semi-log compound dilution series. IC₅₀ calculations are performed using Excel and XLf it (Microsoft), and this value is used to determine a Kᵢ value for the test compound using the Cheng-Prusoff equation.

### Biological Results

All compounds of the Examples above were tested in the CRTH2 radioligand binding assay described above; the compounds had a Kᵢ value of less than 1 μM in the binding assay. For example, Example 1 had a Kᵢ value of 66 nM.

1. A compound which is an indole derivative of formula (I): 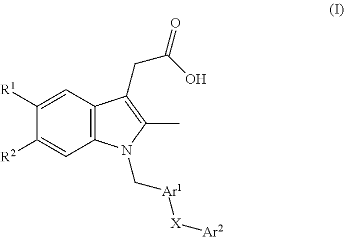
X is —SO₂— or *—SO₂NR³— wherein the bond marked with an asterisk is attached to Ar¹;
R¹ and R² are, independently, hydrogen, fluoro, chloro, CN or CF₃;
R³ is hydrogen, C₁-C₈alkyl or C₃-C₇cycloalkyl; and
Ar¹ and Ar² are, independently, phenyl or a 5- or 6-membered heteroaryl group, wherein the phenyl or heteroaryl group is optionally substituted by one or more substituents independently selected from fluoro, chloro, CN, C₃-C₇cycloalkyl, —O(C₁-C₄alkyl) or C₁-C₆alkyl, the latter two groups being optionally substituted by one or more fluoro atoms.

2. The compound as claimed in claim 1 wherein R² is hydrogen and R¹ is fluoro.

3. The compound as claimed in claim 1 wherein R² is fluoro and R¹ is hydrogen.

4. The compound as claimed in claim 1, wherein ring Ar¹ is a phenyl ring.

5. The compound as claimed in claim 1 wherein ring Ar² is a phenyl ring.

6. The compound as claimed in claim 1 wherein optional substituents in Ar¹ and Ar² are selected from chloro, fluoro, —CN, methyl, ethyl, isopropyl, cyclopropyl, trifluoromethyl, methoxy, isopropoxy, cyclopropoxy, and trifluoromethoxy.

7. The compound as claimed in claim 1 wherein the radical Ar²X— is in the ortho-position of the ring Ar¹ relative to the point of attachment of Ar¹ to the rest of the molecule.

8. A pharmaceutical composition comprising a compound as claimed in claim 1 and a pharmaceutically acceptable carrier.

9-10. (canceled)

11. A method of treatment of asthma, chronic obstructive pulmonary disease, rhinitis, allergic airway syndrome, or allergic rhinobronchitis, comprising administering to a patient suffering such disease an effective amount of a compound as claimed in claim 1.

12. A method of treatment of psoriasis, atopic and non-atopic dermatitis, Crohn's disease, ulcerative colitis, or irritable bowel disease, comprising administering to a patient suffering such disease an effective amount of a compound as claimed in claim 1.
